(19) United States
(12) Patent Application Publication
(10) Pub. No.: US 2017/0035977 A1
(43) Pub. Date: Feb. 9, 2017

(54) Sealing Member for a Medical Device

(71) Applicant: Sanofi SA, Vernier (CH)

(72) Inventor: Stefan Mayer, Frankfurt am Main (DE)

(21) Appl. No.: 15/304,104

(22) PCT Filed: Apr. 14, 2015
(86) PCT No.: PCT/EP2015/058091

§ 371 Date: Oct. 14, 2016

(30) Foreign Application Priority Data
Apr. 16, 2014 (EP) 14164964.0

(51) Int. Cl. A61M 15/00

(52) U.S. Cl. CPC A61M 15/0065; A61M 15/0025; A61M 15/0091; A61M 2202/064; A61M 2207/00

(57) ABSTRACT

The present disclosure relates to a sealing member for an inhaler and an assembly comprising the sealing member. The sealing member comprises a sealing lip. The assembly for an inhaler comprises a first part, a second part, and the sealing member which comprises the sealing lip, wherein the first part and the second part are assembled to permit relative rotational movement between the first part and the second part, and wherein the sealing lip sealingly engages a surface of the second part in order to provide a seal between the first part and the second part.

### CROSS REFERENCE TO RELATED APPLICATIONS

The present application is a U.S. National Phase Application pursuant to 35 U.S.C. §371 of International Application No. PCT/EP2015/058091 filed Apr. 14, 2015, which claims priority to European Patent Application No. 14164964.0 filed Apr. 16, 2014. The entire disclosure contents of these applications are herewith incorporated by reference into the present application.

### FIELD OF INVENTION

The present disclosure is directed to a sealing member for a medical device, particularly to a sealing member for an assembly for a medical device, and to an assembly for a medical device comprising the sealing member. The medical device may be a drug delivery device. Specifically, the medical device may be an inhaler, for example a dry powder inhaler. The present disclosure is further directed to a method of manufacturing an assembly comprising the sealing member.

### BACKGROUND

It is an object of the present disclosure to provide a sealing member which facilitates provision of an improved assembly for a medical device. Additionally, an assembly and a method for manufacturing an assembly for a medical device should be provided.

These objects are, inter alia, achieved by the subject matter of the independent claims. Advantageous refinements and embodiments are set forth throughout the disclosure and in the dependent claims. The present disclosure, however, is not construed to be limited to the claimed subject matter. For example, if the claims refer to an assembly and a method of manufacturing the assembly, the present disclosure also refers to the sealing member per se without features of the assembly. Accordingly, features which are disclosed in the following text with respect to the assembly should also be regarded as being disclosed with respect to the sealing member per se without referencing one or more further parts of the assembly.

### SUMMARY

An aspect of the present disclosure relates to a sealing member. The sealing member comprises a sealing lip. The sealing lip is preferably flexible. The sealing lip may be elastically deformable. As compared to a comparatively rigid sealing member without a sealing lip, such as an O-ring, for example, a sealing member with a sealing lip may be used to establish a seal between two parts where the area of contact of a surface which is sealingly engaged by the sealing lip is reduced as compared to when an O-ring is used as a sealing member. Nevertheless, the sealing lip may provide for a seal which is at least as tight as the seal provided by the O-ring. A reduction in the contact area is especially suitable if the sealing member is provided to establish a dynamic seal, i.e. a seal between components which move with respect to each other, as the static and dynamic friction between the sealing lip and the surface is advantageously small on account of the small contact area. Consequently, the force which has to be exerted to set the parts in motion relative to one another from a rest position where there is no relative movement can be reduced. The same holds for the force which has to be exerted to keep the parts moving relative to one another. The reduced force is of particular advantage if the force has to be generated by a user, as provision of the sealing lip will result in less effort for the user.

Another aspect relates to an assembly for a medical device, particularly a drug delivery device such as an inhaler. For example, the assembly may be an assembly for a dry powder inhaler. The assembly comprises a first part and a second part. The first part and the second part may be assembled, for example directly coupled or coupled via one or more further part(s). The first part and the second part may be assembled to permit relative movement between the first part and the second part. The relative movement may be relative rotational movement.

The second part may be rotatable relative to the first part or vice versa. The first and the second parts may be assembled such that the first part and the second part are rotatable relative to each other with relative axial movement between the first and the second part being prevented. Consequently, the first and the second parts may be axially secured to one another. The assembly further comprises the sealing member which, in turn, comprises the sealing lip. Preferably, the sealing lip sealingly engages or contacts a surface of the first part or the second part, such as an outer surface of the second part or an inner surface of the first part. By means of the sealing engagement a seal may be provided between the first part and the second part.

On account of the comparatively small contact area between the sealing lip and the surface, the force which has to be overcome to initiate relative movement between the first part and the second part is small, particularly as compared to sealing members without a sealing lip. Preferably, the seal provided by the sealing member is a dynamic seal, like a rotation seal, which is effective during relative movement of the parts, such as during relative rotational movement. The sealing lip may extend within an interspace between the first part and the second part. Particularly, the sealing lip may fill a space between the first part and the second part which would otherwise be an unfilled space. The interface between the first part and the second part may be tightly sealed in the assembly by means of the sealing lip.

The seal provided by the sealing member and, in particular, its sealing lip may be fluid-tight, moisture-tight and/or powder-tight. Moisture-tight and/or powder-tight seals are especially suitable for inhalers which are designed to deliver medicament in powder form, such as dry powder inhalers, because they prevent malfunction of the device due to humid powder and/or constrain dry powder within an area of the assembly, such as a powder reservoir, to which it belongs. Contamination of other areas of the assembly with powder can be avoided or reduced considerably. Specifically, if the seal is provided between two parts which can rotate relative to one another, powder within an interface area of the parts could considerably increase the force which is required to rotate the parts as it increases the friction which has to be overcome.

The first part may define an interior space of the assembly. The first part may be arranged and configured to receive powder, for example in the interior space. The powder reservoir may be provided by or in the interior space. The second part may be arranged and configured to retain a dosing member which is arranged to retrieve a sub-quantity of powder from the first part. The dosing member may be movably retained within the second part. Preferably, the assembly is configured such that, when the first part is rotated relative to the second part, a sub-quantity of powder is transferred from the first part into the second part by means of the dosing member.

In an embodiment, the sealing member is connected to one of the first part and the second part and, preferably, not, in particular not rigidly, connected to the other one of the first part and the second part. The sealing lip may contact a surface of the other one of the first part and the second part, e.g. of that part to which the sealing member is not connected.

In an embodiment, the sealing lip defines an opening. The opening may be circumferentially delimited by the sealing lip. A section of the second part may be received within the opening and sealingly engaged, in particular circumferentially, by the sealing lip. An inner diameter of the opening of the unassembled sealing member may be smaller than an outer diameter of the section of the second part. During assembly, the section is introduced into the opening and the sealing lip is deformed due to the greater diameter of the section.

In an embodiment, the sealing member is formed ring-like. The lip may define the inner diameter of the ring.

In an embodiment, the sealing lip exerts a sealing force onto the surface of the second part or the first part. The sealing force may comprise or consist of a radial component. Alternatively, the sealing force may comprise or consist of an axial component. In an embodiment, the sealing force may comprise both a radial component and an axial component. The directional specifications like “radial” and “axial” may refer to the rotation axis around which the second part is rotatable relative to the first part or vice versa. The elastic deformation may take place in the axial and/or radial direction. Thus, the sealing lip may be radially and/or axially deflected or deformed when the assembly is manufactured.

In an embodiment, the sealing lip is deformed, preferably elastically deformed, for example in account of the sealing engagement with the surface. An elastic deformation of the sealing lip may provide for an elastic restoring force, which tends to move the sealing lip into its undeformed or original shape. This force is counteracted by the surface of the first or second part. Consequently, the restoring force acts on the surface. Thus, the sealing force which is exerted via the sealing lip onto the first part or the second part may be or may comprise the elastic restoring force. The radial and axial components of the sealing force may be exerted onto the surface and/or transferred to the surface by the elastically deformed sealing lip.

In an embodiment, the sealing lip comprises a free end. The free end may be a pointed end. In the sealing engagement, the sealing lip may be deformed, preferably only in an end section of the sealing lip adjacent to the free end.

In an embodiment, the sealing member comprises a main body. The sealing lip may project from the main body. The sealing lip and the main body may be formed unitarily. The free end of the sealing lip may be arranged on that side of the sealing lip which is remote from the main body. The main body may define an opening, for example the main body may be formed ring-like. The sealing lip may protrude inwardly from an interior surface of the main body. As compared to the sealing lip, the main body may be more rigid. Consequently, the main body may be used to connect the sealing member to a part. The sealing lip is expediently provided to perform the sealing action of the sealing member. The main body, preferably, does not take part in the sealing action.

In an embodiment, the sealing member is connected to the first part. Particularly, the main body may be connected to the first part. The main body may be used to connect the sealing member stably to the first part. The sealing member and the first part may be formed integrally. For example, the sealing member and the first part may be comprised by a single 2K-molded component. For example, the first part may be more rigid than the sealing member. An integral formation of the sealing member and the first part integrates the sealing member into the first part and offers an easy production method.

In an embodiment, the sealing lip tapers in a direction towards the free end. Consequently, as seen in a sectional view, for example taken along the rotation axis, the width of the sealing lip may decrease towards the free end. As seen from the free end, the width may increase. In a section near the free end, preferably in the section immediately adjoining the free end, the sealing lip may exhibit an asymmetrical cross section as seen along the main direction of extent of the sealing lip.

In an embodiment, the sealing lip comprises two lip surfaces, which may delimit the sealing, particularly along its main direction of extent. The two lip surfaces may extend towards the free end. The two lip surfaces may originate in the main body. The two lip surfaces may extend parallel or essentially parallel with respect to each other towards the free end. The free end of the sealing lip may be arranged closer to one of the lip surfaces than to the other one of the lip surfaces. The two lip surfaces may define the radial extension of the sealing lip. One of the lip surfaces may lead directly to the free end and the other one of the lip surfaces may be connected to the free end by an oblique surface, preferably an oblique end surface. The respective surfaces may be plane surfaces. The lip surface which leads directly to the free end may be in mechanical contact with the surface of the second part to establish the seal. Accordingly, the oblique surface may be remote from the surface of the second part.

By means of an appropriate design of the sealing lip—with reduced cross section in the end section and/or by the oblique surface—, it can be ensured that the sealing force acting on the surface of the first or second part is as small as possible but still provides for a tight seal.

In an embodiment, the sealing lip is designed and preferably arranged, for establishing a seal, to be deflected in a direction away from that lip surface which is closer to the free end or which leads directly to the free end.

In an embodiment, the sealing lip is designed to define a cavity when deformed. Particularly, the deformed sealing lip may define the cavity. The cavity may be arranged on that side of the deformed sealing lip which faces away from the surface of the second part. Particularly, the cavity may be delimited, at least partly, by the sealing lip and by the main body and/or by the sealing lip and the first part.

In an embodiment, the first part or the second part defines an interior space. The powder reservoir may be provided in or by the interior space. Preferably, powder is retained in the powder reservoir. An interior of the powder reservoir may be in fluid communication with the cavity. Particularly, the fluid communication may be established such that powder is allowed to enter the cavity. In other words, the cavity may be arranged and configured to receive powder. The cavity and the fluid communication may be arranged and configured such that enough powder may enter the cavity to transfer a force, expediently a significant force, to the sealing lip via the powder. For this purpose, the powder may be compacted and subjected to an additional force, e.g. a spring force, which, on account of the compact powder, is transferred to the sealing lip via the powder. Accordingly, the force transferred by the powder may advantageously contribute to the sealing force which is exerted on the surface of the first or second part via the sealing lip. Thus, the powder itself may be used to establish or strengthen the seal. When the first and second parts are assembled, the force required to deform the sealing lip may be smaller than the sealing force which later on acts on the surface, because the powder has usually not yet been introduced into the powder reservoir in that stage of the assembling process. After the powder has been introduced and the additional force has been applied, the seal may be strengthened. It should be readily apparent that the disclosed concepts would also work for liquid medical substances and not only for powder.

In an embodiment, the assembly comprises a movable wall, preferably a biased movable wall. The movable wall may be biased by a spring, expediently a preloaded spring. The movable wall may delimit the interior of the powder reservoir. The movable wall may be a trailing wall, which is operable to reduce the volume of the powder reservoir continuously when powder is removed from the reservoir, for example during dosing. The movable wall may keep the powder compacted such as by transferring the spring force to the powder. As the wall is movable, powder within the reservoir may be kept compacted by biasing the movable wall, in particular regardless of the amount of powder remaining in the reservoir. Preferably, the powder within the reservoir is kept under mechanical pressure by the bias acting on the movable wall. The powder within the reservoir may be subject to a force which is exerted by the movable wall onto the powder. This force may be transferred to the sealing lip via the powder and to the surface of the first or second part via the sealing lip. Consequently, the force which biases the movable wall may be used to establish the seal. Particularly, the tightness of the seal may be increased by means of the powder.

In an embodiment, the first part or the second part, expediently the one which does not define the interior space recited above, comprises an opening which is in fluid communication with the powder reservoir. A dosing member, e.g. a metering rod comprising a metering chamber, of the assembly may be arranged and configured to be movable to retrieve a sub-quantity of powder from the powder reservoir and to transfer the sub-quantity from the reservoir into the second part, particularly through the opening. The dosing member may be moveably retained in the second part. When the sub-quantity is transferred from the reservoir into the second part, the first part and the second part may rotate relative to one another. The rotation axis may extend through the opening. The opening in the second part may be a central opening. The sealing member may be further away from the rotation axis than the opening.

A further aspect relates to a medical device, preferably a drug delivery device such as an inhaler, which comprises an assembly, particularly the assembly described above and further below. Accordingly, features described in conjunction with the sealing member or the assembly above and further below do also apply to the medical device and vice versa.

A further aspect relates to a method of manufacturing an assembly for a medical device such as a drug delivery device, particularly an inhaler. The assembly may be the assembly as described above and further below. Accordingly, features disclosed above and below in connection with the assembly, the sealing member or the inhaler, also apply to the method and vice versa.

In the method, a first part is provided. A sealing member which comprises a sealing lip is provided. The sealing member may be connected to the first part. The sealing lip may define an opening having a first diameter. The first diameter may be the inner diameter of the sealing member. The sealing lip may have a free end. Also, a second part may be provided, the second part having a section which has a second diameter, particularly an outer diameter, which is greater than the first diameter. The section of the second part may be guided into the opening defined by the sealing lip. The guiding may be performed such that the sealing lip contacts the section, particularly its outer surface, thereby deforming the sealing lip such that an end section, e.g. a section immediately adjoining the free end, of the sealing lip extends along the section of the second part. The end section of the sealing lip may be deflected on account of its contact with the section of the second part. Afterwards, the first and the second part may be rotatably secured to each other. The rotatable securing may be performed either by directly securing the first part and the second part rotatably to each other or via a further part which is rotationally locked to one of the first and second parts and rotatably secured to the other one of the first part and the second part.

The method has the advantage that the first and the second parts may be assembled in an easy way with a seal being established by the deformation of the sealing lip during the assembling process. No further steps have to be performed for providing the seal. Furthermore, the part count is low as the sealing lip may be integrally formed with the first part.

In the following text, some aspects of this disclosure are described. The aspects are numbered in order to facilitate referencing the features of certain aspects in other aspects,

1. Assembly for an inhaler, comprising:
a first part (3)
a second part (25), and
a sealing member (90) which comprises a sealing lip (92), wherein
the first part and the second part are assembled to permit relative rotational movement between the first part and the second part, and wherein
the sealing lip sealingly engages a surface of the second part in order to provide a seal between the first part and the second part.

2. Assembly according to aspect 1,

wherein the sealing lip (92) exerts a sealing force, which comprises radial and axial components, onto the surface of the second part (25).

3. Assembly according to any one of the preceding aspects,

wherein the sealing member (90) comprises a main body (91), the sealing lip (92) projecting from the main body, and wherein the sealing lip comprises a free end (93) on that side of the sealing lip which is remote from the main body.

4. Assembly according to aspect 3,

wherein the main body (91) is connected to the first part (3).

5. Assembly according to aspect 3 or 4,

wherein the sealing lip (92) tapers in a direction towards the free end (93).

6. Assembly according to any one of the preceding aspects,

wherein the sealing lip comprises two lip surfaces (94, 95) which extend towards the free end (93), one of the lip surfaces (94) leading directly to the free end and the other one of the lip surfaces (95) being connected to the free end by an oblique surface (96).

7. Assembly according to aspect 6,

wherein the lip surface (94) which leads directly to the free end (93) is in mechanical contact with the surface of the second part (25).

8. Assembly according to any one of the preceding aspects,

wherein the sealing lip (92), on account of the sealing engagement with the surface of the second part (25), is elastically deformed.

9. Assembly according to aspect 8,

wherein the deformed sealing lip (92) defines a cavity (101), the cavity being arranged on that side of the deformed sealing lip which faces away from the surface of the second part (25).

10. Assembly according to aspect 9,

wherein the cavity (101) is arranged and configured to receive powder.

11. Assembly according to aspect 9 or 10, wherein the first part (3) defines an interior space, a powder reservoir (15) being provided in or by the interior space, wherein an interior of the powder reservoir is in fluid communication with the cavity (101).

12. Assembly according to aspect 11,

wherein the assembly comprises a biased movable wall (80), which delimits the interior of the powder reservoir (15), and wherein powder (2) is retained within the reservoir, wherein the powder within the reservoir is subject to a force which is exerted by the movable wall onto the powder, this force being transferred to the sealing lip (92) via the powder and to the surface via the sealing lip.

13. Assembly according to aspect 11 or 12,

wherein the second part (25) comprises an opening which is in fluid communication with the powder reservoir, wherein a dosing member of the assembly is arranged and configured to be moveable to retrieve a sub-quantity (14) of powder from the reservoir (15) and to transfer the sub-quantity from the reservoir into the second part through the opening.

14. Inhaler (1) comprising the assembly according to any of the preceding aspects.

15. Method of manufacturing an assembly for an inhaler according to any one of the previous aspects 1 to 13,

comprising the following steps:
Providing a first part (3), a sealing member (90) which comprises a sealing lip (92) being connected to the first part, wherein the sealing lip defines an opening having a first diameter;
providing a second part (25), the second part having a section (842) which has a second diameter which is greater than the first diameter;
guiding the section of the second part into the opening, such that the sealing lip contacts the section, thereby deforming the sealing lip such that an end section of the sealing lip extends along the section of the second part;
rotatably securing the first part and the second part to each other.

### BRIEF DESCRIPTION OF THE DRAWINGS

Further advantages, expediencies and refinements of the present disclosure will become apparent from the following description of the exemplary embodiment in conjunction with the figures.

FIG. 1 shows an embodiment of an inhalation device on the basis of a schematic sectional view.

FIG. 2 shows an embodiment of a sealing member based on a schematic sectional view.

FIG. 3A shows an embodiment of an assembly in a not fully assembled condition and FIG. 3B illustrates a detail of the assembly in an assembled condition.

### DETAILED DESCRIPTION

In the figures, identical elements, elements of the same kind and identically acting elements may be provided with corresponding reference numerals. Furthermore, certain sections of the figures may be depicted in an exaggerated fashion and not true to scale to facilitate explanation of the disclosed concepts.

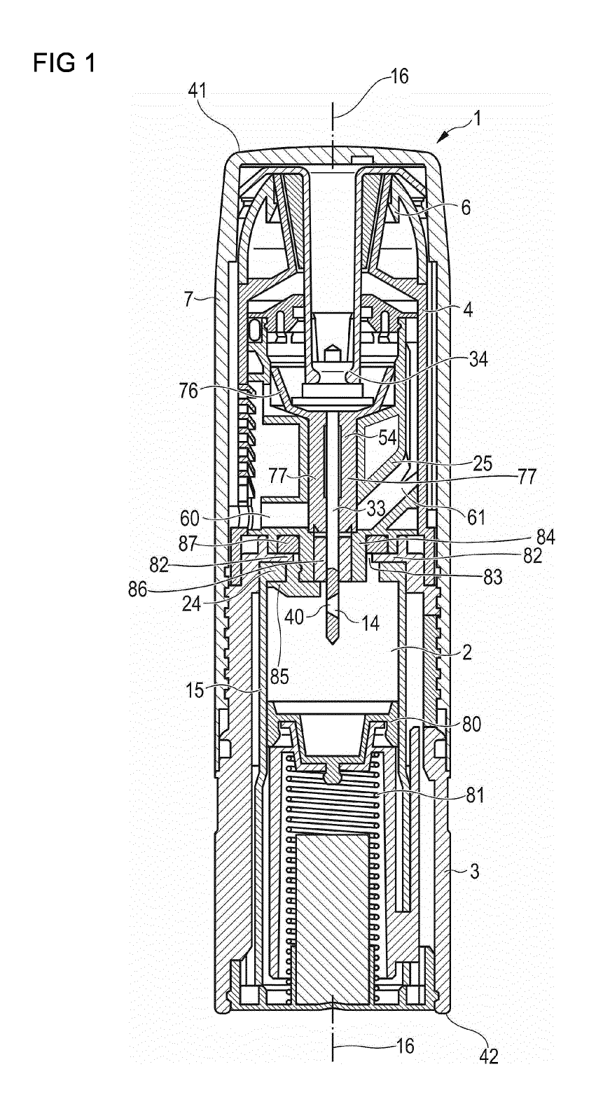

In FIG. 1, a sectional side view of an inhalation device 1 is shown. The inhalation device 1 comprises a housing 3. The device 1 comprises an outer part or outer cylinder 4. Whenever reference is made to the outer cylinder below, this may also be regarded as referring to the outer part. The part is denoted as outer part although it is not arranged on the outside as depicted in FIG. 1, because this part defines an outer surface of the device when the device is used to dispense a dose of drug. The outer cylinder 4 is secured against axial movement with respect to the housing 3. The outer cylinder 4 is rotatable with respect to the housing 3. The inhalation device 1 further comprises a mouthpiece 6, which may be fixedly connected to the outer cylinder 4. The device 1 and the housing 3 have a distal end 41 and a proximal end 42. The term “distal end” designates that end of the device 1 or a component thereof which is located or is to be arranged closest to the mouthpiece 6. The term “proximal end” designates that end of the device 1 or a component thereof which is located or is to be arranged furthest away from the mouthpiece 6. The distal end 41 and the proximal end 42 are spaced apart from one another in the direction of an axis 16. The axis 16 may be the main longitudinal axis or the rotational axis of the device 1 around which components of the device rotate during operation of the device, as will be further explained below.

The inhalation device 1 comprises a cap 7. The cap 7 is used for covering the mouthpiece 6. The cap 7 may comprise a thread, preferably a screw thread. The cap is preferably threadedly engaged with the housing 3. The cap 7 may be rotatable with respect to the housing 3 for screwing the cap 7 onto the device 1 and for unscrewing the cap 7 from the device 1. The outer cylinder 4 is rotationally fixed to the cap 7. In particular, the outer cylinder 4 follows rotation of the cap 7 with respect to the housing 3. For this purpose, a splined interface may be provided between the cap and the outer cylinder (not explicitly illustrated). For the detailed description of the components of the inhalation device 1 and their mechanical cooperation, reference is made to document WO 2009/065707 A1, which shows a similar device. The disclosure of this document is hereby explicitly incorporated into the present disclosure by reference.

The device 1 comprises a storage chamber 15. The storage chamber is retained within the interior of the housing 3. The storage chamber 15 holds at least one dose, preferably a plurality of doses, of a medical substance 2. The substance 2 may be or may comprise a powder. The storage chamber 15 may therefore constitute a powder reservoir. The medical substance may comprise a drug.

The term “drug” as used herein may mean a pharmaceutical formulation containing at least one pharmaceutically active compound, for example for the treatment of obstructive airway or lung diseases such as asthma or chronic obstructive pulmonary disease (COPD), local respiratory tract oedema, inflammation, viral, bacterial, mycotic or other infection, allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));

a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);

a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

a phosphodiesterase type-4 inhibitor (e.g. Roflumilast);

an antihistamine (e.g. Loratadine, Desloratadine, Cetirizen, Levocetirizine, Fexofenadine);

an allergen immunotherapy (e.g. Omalizumab);

a mucolytic (e.g. Carbocisteine, Erdosteine, Mecysteine);

an antibiotic or antimycotic;

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

The storage chamber 15 is terminated on one end by a chamber ceiling 24. The chamber ceiling 24 is formed integrally with a top wall of the storage chamber 15. Through an opening within the chamber sealing 24, the metering rod 33 can enter the storage chamber 15 and retrieve substance from the storage chamber.

At another end of the storage chamber 15, e.g. an end opposite to the opening, a movable wall 80 is arranged. The movable wall 80 delimits the interior of the storage chamber 15. The movable wall 80 is biased by a spring 81. The movable wall 80 is biased towards the opening through which the metering rod 33 may enter the storage chamber 15. By means of the biased movable wall 80 the substance within the storage chamber 15 can be kept compact or compacted even if several doses have been retrieved from the chamber by means of the metering rod 33. Accordingly, it can be guaranteed that there is always enough substance available for a metering operation.

The device 1 further comprises a rotary part 25. The rotary part 25 is connected in a rotationally and preferably axially fixed manner to the outer cylinder 4. Accordingly, the rotary part 25 follows rotation of the cap 7 and, hence, of the outer cylinder 4 about the main longitudinal axis or rotational axis 16 of the device 1 with respect to the storage chamber 15. However, the rotary part 25 is axially fixed relative to the housing 3, for example via the outer cylinder 4 which is rotatably secured to the housing. Alternatively, the rotary part may be rotatably secured to the housing 3. In some embodiments, the outer cylinder 4 and the rotary part 25 may be formed unitarily.

The device 1 further comprises a metering rod 33. The metering rod comprises a metering chamber 40. The metering rod 33 is movably retained within the rotary part 25. The metering rod is axially guided within the rotary part 25 such that no relative rotation is performed between these parts. Accordingly, the metering rod 33 follows rotational movement of the cap 7 and, hence, of the rotary part 25 about the main longitudinal axis 16 when the cap 7 is screwed or mounted onto the device 1 or unscrewed or demounted from the device 1. The metering chamber 40 is axially movable relative to the rotary part 25 from a position inside the storage chamber 15 into a position outside the storage chamber in order to retrieve a dose of powder from the chamber 15 and move it into a position within the rotary part in which position the dose is ready for inhalation. The metering rod 33 may be connected to the cap 7 by a snap fit element 34 when the cap 7 is engaged to the housing 3.

The housing 3 has an inwardly, e.g. radially inwardly, directed portion 82, particularly a shoulder portion. The portion 82 may be arranged on that end of the housing which faces the rotary part 25. The housing 3 furthermore comprises an opening 83 through which the metering rod 33 may extend from outside the storage chamber 15 into the storage chamber. The opening 83 may be defined and/or delimited by the portion 82. A section 84 of the rotary part 25, preferably an end section, which faces the housing 3 extends through the opening 83. The section 84 may also extend through the opening of the storage chamber 15. The section 84 may be a cylindrical section of the rotary part.

An organizing member 85 is locked, preferably axially and rotationally, to the rotary part 25, and in particular to its section 84. The organizing member is arranged within the interior of the storage chamber and, during operation of the device, is in direct contact with the powder. As the rotary part 25 rotates, the organizing member 85 also rotates and agitates the powder in the chamber. Thus, the organizing member 85 ensures that the powder is kept loose and no unwanted powder agglomerations can form within the storage chamber 15. Within the interior of the rotary part 25, particularly within the interior of the section 84, a seal 86 is arranged which removes excessive powder sticking to the metering rod 33 while the metering rod moves through that seal from the interior of the storage chamber 15 into the rotary part 25 to the position in which it is ready for inhalation, which is approximately level to reference numeral 60 in FIG. 1.

When the cap 7 is disengaged from the housing 3, the metering rod 33 travels axially in the distal direction such that the most proximal part of the metering rod 33 exits the storage chamber 15. The metering chamber 40 is provided in that end section of the metering rod 33 which projects into the substance 2, e.g. the proximal end section. When the cap 7 is re-engaged to the housing 3, the metering rod 33 travels axially in the proximal direction such that a most proximal part of the metering rod 33 comprising a metering chamber 40 re-enters the storage chamber 15.

The inhalation device 1 further comprises a flow path comprising a flow channel 60 and an intermediate channel portion 61, which are expediently fluidly connected to the mouthpiece.

The inhalation device 1 further comprises a closure element 54. The closure element 54 is arranged within the rotary part 25 and movably guided in that part. The rotary part 25 and the closure element are arranged to co-rotate. Accordingly, no net rotation takes place between these parts during operation.

The closure element 54 may be formed by a piston comprising tongues 77 and a head 76. The head 76 of the closure element 54 is formed from a soft material which can be deformed easily.

The closure element 54 has a first and a second position. The first position is more proximal than the second position. In the first position, the closure element 54, particularly the tongues 77 of the closure element 54, is configured to block the flow path between the flow channel 60 and the intermediate channel portion 61 (this position is depicted in FIG. 1). In the second position, the closure element 54 is positioned more distally, i.e. closer to the mouthpiece 6, such that the tongues 77 no longer block the flow path between the flow channel 60 and the intermediate channel portion 61.

The cap 7 is removed from the housing 3 by unscrewing the cap 7 from the housing 3. Accordingly, the cap 7 performs a concurrent axial movement in the distal direction and a rotational movement. The cap 7 and the outer cylinder 4 are in a splined engagement when the cap 7 is attached to the housing 3. During disengagement of the cap 7 from the housing 3, the rotational movement of the cap 7 is transferred into a rotation of the rotary part 25 around the longitudinal axis 16 due to the splined coupling to the outer cylinder. The rotation of the rotary part 25 is transferred into a rotation of the closure element 54. Furthermore, the concurrent axial and rotational movement of the cap 7 is transferred to the metering rod 33 concurrently performing an axial movement in the distal direction relative to the storage chamber 15 and the rotary part 25 and a rotational movement around the longitudinal axis 16. As the cap 7 approaches the end of the threaded connection to the housing 3, the snap fit element 34 disengages from the metering rod 33. During disengagement of the cap 7 from the housing 3, the closure element 54 is not moved in an axial direction with respect to the housing 3. Accordingly, the closure element 54 is in the first position before and after disengagement of the cap 7 from the housing 3.

When the cap 7 is fully disengaged from the housing 3, the metering chamber 40 has been moved from the storage chamber into a position within the flow path (not explicitly shown). Specifically, the metering chamber 40 is then arranged between the channels 60 and 61. In this position, the tongues 77 of the closure element 54 close the metering chamber 40 such that the metering chamber 40 is not exposed to the flow path. Accordingly, when the closure element 54 is in the first position and the cap 7 has been disengaged from the housing 3, the tongues 77 of the closure element 54 cover the metering chamber 40 on both sides. Accordingly, in this first condition, it is not possible for the sub-quantity 14 of substance to trickle out. Rather, the substance 2 is reliably retained in the metering chamber 40.

After the cap 7 has been unscrewed, the user may trigger an inhalation operation by subjecting the device to a suction airstream, in the simplest case by the user breathing in. Air is sucked in via the mouthpiece 6, and this, e. g. by virtue of the head 76 being subjected to the action of air, results in the closure element 54 being displaced axially towards the mouthpiece 6, i.e. in the distal direction. By virtue of the axially displaced closure element 54, the tongues 77 are likewise displaced axially, in order to open the metering chamber 40. The metering chamber 40 then lies freely in the flow path between the flow channel 60 and the intermediate channel portion 61. Then the metering chamber 40 is emptied and the sub-quantity 14 is carried towards the mouthpiece 6 by the air which is sucked in through the flow channel 60.

Once the inhalation operation has been completed, the cap 7 can be re-engaged to the housing 3. During engagement of the cap 7 to the housing 3, the cap 7 is moved axially in the proximal direction and concurrently rotated around the longitudinal axis 16. The snap fit element 34 engages to the metering rod 33 at the beginning of the threaded connection. Thereby, the metering rod 33 is moved into the proximal direction when the cap 7 is engaged to the housing 3. Movement of the metering rod 33 in the proximal direction is transferred to the closure element 54 (if the closure element 54 is in the second position). As a result, the closure element 54 is moved from the second position to the first position. However, in the case that the cap 7 is disengaged from the housing 3 and, afterwards, re-engaged to the housing 3 without a drug delivery being performed in the meantime, the closure element 54 remains in the first position the whole time. Accordingly, as the closure element 54 is already in the first position, it cannot be moved axially during engagement of the cap 7 to the housing 3.

In the embodiment shown in FIG. 1, a sealing ring 87 in the form of an O-ring is arranged at the interface of the rotary part 25 and the housing 3. The ring may be inserted with radial preload in a notch of the rotary part, as depicted, such that it is reliably retained in the notch when the rotary part rotates relative to the housing 3. The sealing ring 87 may also contact the housing 3, particularly housing portion 82. Consequently, the sealing ring provides a seal which prevents powder from getting into the rotary part and moisture from getting into the storage chamber even if the rotary part 85 rotates relative to the housing 3.

The concepts disclosed below are directed towards replacing the sealing ring 87 with a different sealing member which has advantages and features which are set forth below and also in the introductory section of the application. Not all features described in the introductory section are necessarily repeated below but nevertheless apply to the disclosed concepts. Even though the present sealing concept is described in conjunction with the rotary part 25 and the housing 3 as exemplary embodiments of two parts, it may also apply to other parts in different assemblies or for medical devices, such as inhalation devices.

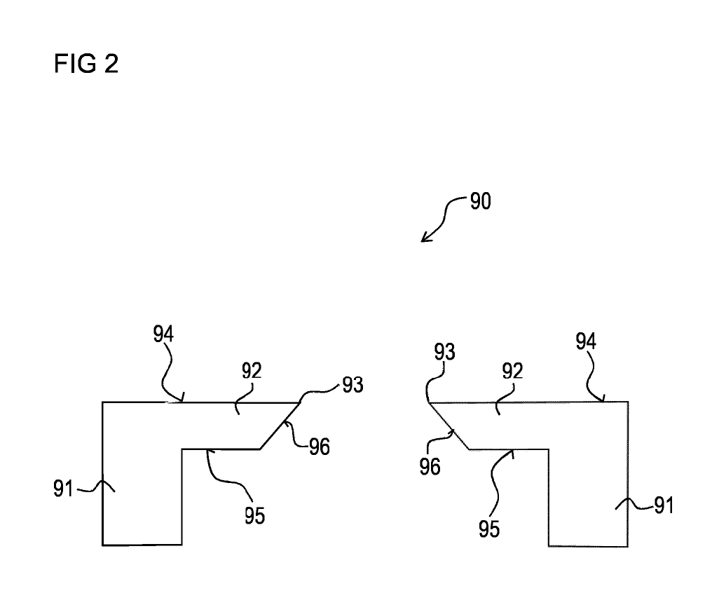

FIG. 2 shows an embodiment of an advantageous sealing member which can be applied in the device described in conjunction with FIG. 1 and has advantageous effects for the device operation which has already been described above. FIG. 2 shows the sealing member 90 in a schematic sectional view.

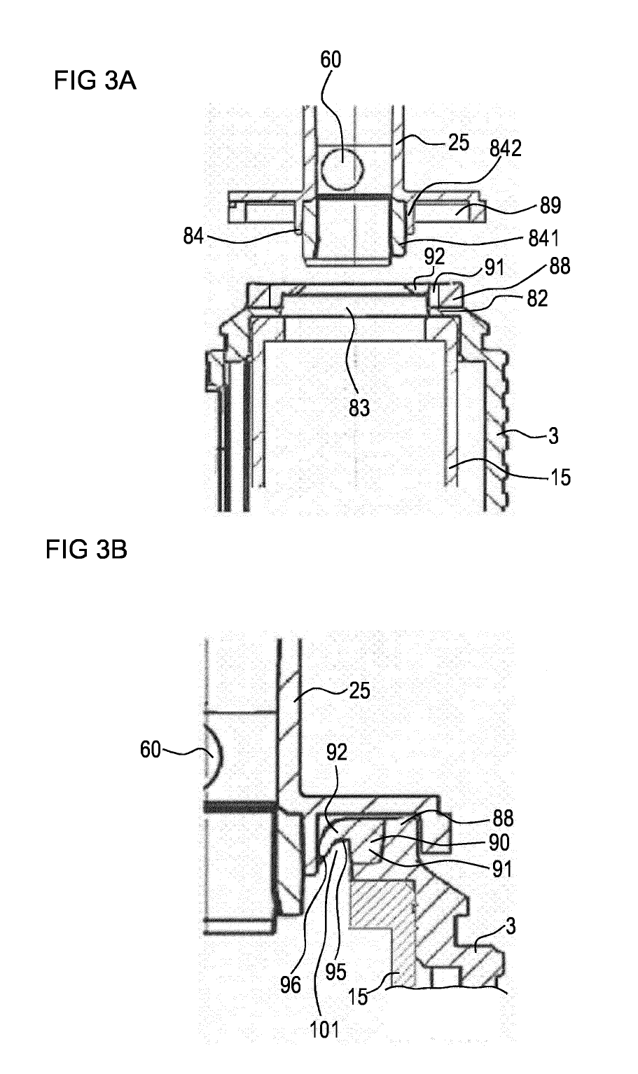

The sealing member 90 has a main body 91. The sealing member 90 is of a generally ring-like construction. The sealing member defines a central opening. The sealing member 90 comprises a sealing lip 92. The sealing lip 92 protrudes inwardly from the main body. The lip 92 can, as compared to the main body 91, be elastically deformed more easily. The sealing lip 92 and the main body 91 are, preferably, rotationally symmetrically disposed around an axis extending through the opening. The opening may be delimited circumferentially by the sealing lip 92. The sealing member may be formed rotationally symmetrically with respect to this axis. The sealing lip 92 comprises a free end 93. The free end 93 is disposed on the side of the sealing lip which is remote from the main body 92. The free end 93 may be a pointed free end. The sealing lip 92 is defined by two lip surfaces, a first lip surface 94 and a second lip surface 95. Lip surfaces 94 and 95 may be arranged such that they largely extend along each other, for example they may be arranged parallel to each other. The lip surfaces 94 and 95 may extend in the radial direction, particularly with respect to the main body. A radial extension of the first lip surface 94 may be greater than that of the second lip surface 95. The second lip surface 95 may be connected to the free end 93 by an oblique surface 96, e.g. a surface which is neither perpendicular nor parallel to the axis, which may be the axis around which the rotation in the device is performed when the sealing member is incorporated in the device. The oblique surface 96 may connect, particularly directly connect, the second lip surface 95 to the free end 93. The first lip surface 94 may lead directly to the free end. Particularly, the first lip surface 94 may continue a surface of the main body in an even fashion without interruption, as depicted. Alternatively, the first lip surface may be axially recessed with respect to the surface of the main body. The second lip surface may be radially offset from the free end 95. For establishing a sealing engagement, the sealing lip is expediently configured such that the free end is axially deflectable into that direction into which the second surface 95 is offset from the free end, i.e. downwards in FIG. 2. As the sealing lip 92 tapers in the end section, i.e. its width decreases in cross-section towards the free end, a deflection of this kind requires only advantageously low forces. Consequently, the force exerted on the sealing member during an assembling process can be kept advantageously small. The main body 91 and the sealing lip 92 are expediently formed unitarily. For example, the sealing member may comprise or consist of a TPE, i.e. a thermoplastic elastomer. Thermoplastic elastomers exhibit good elasticity, which is required for an efficient seal, particularly a seal established by a sealing lip which is deformed to establish the sealing engagement. The sealing lip 91 further extends radially to such an extent that, in a deformed state, when it is in sealing engagement, it defines a cavity which is delimited by the second lip surface and the main body (see cavity 101 in FIG. 3B).

FIGS. 3A and 3B illustrate an advantageous assembly which may be implemented in the device described in conjunction with FIG. 1 during two different states of manufacture. In FIG. 3A the assembly is still unassembled, whereas FIG. 3B shows a detail of the assembled assembly.

In FIG. 3A the housing 3 is provided together with the storage chamber 15 retained in the interior of the housing 3. The sealing member 90 is connected, preferably rotationally and axially secured to the housing 3. Particularly, the sealing member 90 may be arranged on, and preferably in direct contact with, the housing portion 82. The main body 91 of the sealing member 90 may bear on the housing portion 82. The sealing member, particularly its main body, may be in direct contact with an axially protruding housing portion 88 such that a reliable seat is formed for the sealing member. Advantageously, the sealing member 90 and the housing 3 may be formed integrally, for example by a 2K molding process where the sealing member is molded to the housing body 3. The housing body 3 may, for example, comprise or consist of PP (polypropylene).

As depicted in FIG. 3A, the section 84 is arranged to be guided through the opening defined by the sealing member 90. The section 84 has two sub-sections, 841, 842. Sub-section 841 may be formed by a part attached to a main body of the rotary part 25 or may be formed integrally with the rotary part 25. Sub-section 841 faces the opening defined by the sealing member and is followed by sub-section 842 in a direction away from the opening. Sub-section 841 may have an outer diameter which is smaller than that of sub-section 842. The diameter of sub-section 841 may be even smaller than the diameter of the opening. The diameter of sub-section 842, in contrast, is greater than that of the opening in the sealing member. The rotatory body 25 further comprises a notch 89 which faces the housing 3 and is configured to retain the sealing member 90 as well as a portion of the housing, such as housing portion 88.

From the position shown in FIG. 3A, the rotary part 25, and particularly section 84, is guided into the opening defined by the sealing member 90. At first, when sub-section 841 enters the opening, no or no significant deformation of the sealing lip 92 occurs. When the section with a larger outer diameter, i.e. section 842, enters the opening, the sealing lip 92 is deformed in the axial and radial direction such that the sealing lip is bent downward as well as outward. Specifically, when section 84, particularly sub-section 842, enters the opening, the pointed free end 93 contacts section 84 of the rotary part 25 and conforms to the outer surface of the rotary part 25 and, consequently, on account of the elastic restoring force, exerts a sealing force onto this surface. The sealing force prevents moisture from the outside from entering the storage chamber. The powder within the storage chamber may enhance the sealing effect (see below). Particularly, the higher the force exerted by the powder onto the sealing lip, the better the sealing effect.

Relative axial movement between the parts, i.e. the rotary part 25 and the housing 3, is stopped when the surface of the rotary part defining the notch engages the portion 88 of the housing 3. A detail of a sectional view illustrating this position is shown in FIG. 3B.

When the parts are in this position, they may be axially fixed to each other, for example by a snap-fit connection or by a further part like the outer cylinder 4, which is not explicitly illustrated in the drawings but may be nevertheless provided. In the position depicted in FIG. 3B, the sealing member 90, including the main body 91 and the deformed sealing lip 92, is arranged within notch 89, particularly together with housing portion 88. Further, the sealing lip 92, in cooperation with the main body 91 and/or a section of the housing 3, defines a cavity 101. The cavity is in fluid communication with the powder which has been filled into the storage chamber 15. As the movable spring-loaded wall 80, which delimits the storage chamber, presses the powder within the reservoir towards the opening in the storage chamber, this powder also enters the cavity 101, particularly in a substantial amount and, consequently, the spring force of the spring 81 is transferred to the sealing lip and thus contributes to the sealing force acting on the outer surface of the rotary part, particularly the outer surface of section 84 and, in this section, on the outer surface of sub-section 842. In this way the spring force may be used to enhance the sealing effect provided by the sealing member.

The contact area of the sealing lip with the rotary part 25 is advantageously small, such that the friction which has to be overcome for rotating the rotary part 25 relative to the housing 3 is advantageously small. The sealing effect is not significantly decreased as compared to an O-ring type seal as described in conjunction with FIG. 1. Also, the elastic restoring force which tends to move the sealing lip 92 into its original or undeformed position shown in FIG. 2, contributes to the sealing force acting on the surface of the rotary part 25 as well as the spring force.

An assembly of the rotary part and the housing body together with the sealing member can be achieved without any additional tools; in particular, no tools are necessary to deform the sealing member such that the rotary part may be guided through the sealing member. Also, during the assembly, the sealing member is not likely to be damaged even though no additional tools are used.

It should be readily apparent for the person of skill in the art that the sealing member, then expediently with an outwardly extending sealing lip, could, instead of being connected to the first part (housing 3), also be connected to the second part (rotary part 25).

1-12. (canceled)

13. Assembly for an inhaler, comprising:
a first part
a second part, and
a sealing member which comprises a sealing lip, wherein
the first part and the second part are assembled to permit relative rotational movement between the first part and the second part, and wherein
the sealing lip sealingly engages a surface of the second part in order to provide a seal between the first part and the second part,
the sealing lip, on account of the sealing engagement with the surface of the second part, is elastically deformed,
the deformed sealing lip defines a cavity, the cavity being arranged on that side of the deformed sealing lip which faces away from the surface of the second part, and
the cavity is arranged and configured to receive powder.

14. Assembly according to claim 13,
wherein the sealing lip exerts a sealing force, which comprises radial and axial components, onto the surface of the second part.

15. Assembly according to claim 13,
wherein the sealing member comprises a main body, the sealing lip projecting from the main body, and wherein the sealing lip comprises a free end on that side of the sealing lip which is remote from the main body.

16. Assembly according to claim 15,
wherein the main body is connected to the first part.

17. Assembly according to claim 15,
wherein the sealing lip tapers in a direction towards the free end.

18. Assembly according to claim 13,
wherein the sealing lip comprises two lip surfaces which extend towards the free end, one of the lip surfaces leading directly to the free end and the other one of the lip surfaces being connected to the free end by an oblique surface.

19. Assembly according to claim 18,
wherein the lip surface which leads directly to the free end is in mechanical contact with the surface of the second part.

20. Assembly according to claim 13,
wherein the first part defines an interior space, a powder reservoir being provided in or by the interior space, wherein an interior of the powder reservoir is in fluid communication with the cavity.

21. Assembly according to claim 20,
wherein the assembly comprises a biased movable wall, which delimits the interior of the powder reservoir, and wherein powder is retained within the reservoir, wherein the powder within the reservoir is subject to a force which is exerted by the movable wall onto the powder, this force being transferred to the sealing lip via the powder and to the surface via the sealing lip.

22. Assembly according to claim 20,
wherein the second part comprises an opening which is in fluid communication with the powder reservoir, wherein a dosing member of the assembly is arranged and configured to be moveable to retrieve a sub-quantity of powder from the reservoir and to transfer the sub-quantity from the reservoir into the second part through the opening.

23. Inhaler comprising the assembly according to claim 13.

24. Method of manufacturing an assembly for an inhaler according to claim 13,
comprising the following steps:
Providing a first part, a sealing member which comprises a sealing lip being connected to the first part, wherein the sealing lip defines an opening having a first diameter;
providing a second part, the second part having a section which has a second diameter which is greater than the first diameter;
guiding the section of the second part into the opening, such that the sealing lip contacts the section, thereby deforming the sealing lip such that an end section of the sealing lip extends along the section of the second part;
rotatably securing the first part and the second part to each other.
